(19) United States
(12) Patent Application Publication
(10) Pub. No.: US 2025/0213788 A1
(43) Pub. Date: Jul. 3, 2025

(54) MEDICAL INJECTION DEVICE WITH GAS EVACUATION

(71) Applicant: GUERBET, VILLEPINTE (FR)

(72) Inventors: Ludovic ALLARD, MILLERY (FR); Catherine CHOCHOL, CHABANIERE (FR)

(73) Assignee: GUERBET, VILLEPINTE (FR)

(21) Appl. No.: 18/851,521

(22) PCT Filed: Mar. 30, 2023
(86) PCT No.: PCT/EP2023/058398

§ 371 Date: Sep. 26, 2024

(30) Foreign Application Priority Data
Mar. 30, 2022 (EP) 22305403.2

(51) Int. Cl. A61M 5/145; A61M 5/00; A61M 5/14; A61M 5/31; A61M 5/36

(52) U.S. Cl. CPC A61M 5/14546; A61M 5/3146; A61M 5/36; A61M 5/007; A61M 2005/1403

(57) ABSTRACT

An injection device for injecting into a medical tubing a medical liquid from a medical liquid container, said injection device comprising a body, a piston (6) arranged in the body for travelling along the longitudinal direction, said piston (6), an evacuation path traversing the piston (6), and a float (24) configured for floating on the medical liquid and configured for travelling within a cavity (36) along the longitudinal direction between a blocking configuration in which an obturating part (24a) of the float (24) obturates the passage (38) of the evacuation path, thereby closing the evacuation path, and an open configuration in which the obturating part 24a of the float (24) is spaced apart from the passage (38), thereby letting open the evacuation path, wherein the float (24) has a metacenter (M) which is spaced apart from the top of the obturating part (24a) by a distance less than one third of the height of the float (24).

### TECHNICAL FIELD

The present invention generally relates to the injection of medical fluids and, more particularly, to an injection device for injecting into a medical tubing a medical liquid from a medical liquid container.

### BACKGROUND OF THE INVENTION

Injection devices are commonly used for injecting into a medical tubing a medical liquid from a medical liquid container. For example, the injection of medical liquid such as iodinated contrast agent is required in 70% of CT scan diagnosis cases. This injection, in about 70% of cases, is performed using an automated contrast agent injector. Injection tubing is required to connect the automated injector to the patient.

Patent application no. U.S. Ser. No. 13/453,335 (US Pub. No. 20120209111) discloses a bladder syringe for a fluid delivery system which includes a cylindrical body, a cap-bladder assembly, a plunger element disposed in the cylindrical body, and a mounting assembly to secure the cap-bladder assembly to the cylindrical body. The cylindrical body has a distal end and a proximal end and defines a throughbore. The cap-bladder assembly is adapted for connection to the distal end of the cylindrical body, and includes a cap body and a bladder. The cap body defines an interior cavity and a distal discharge conduit and is adapted to engage the distal end of the cylindrical body. A disc-shaped bladder is disposed within the interior cavity and typically includes a central membrane portion. The plunger element is disposed in the throughbore of the cylindrical body and is vented to enable evacuation of the space between the plunger element and the cap-bladder assembly in the cylindrical body.

Patent application no. U.S. Ser. No. 10/986,416 (US Pub. No. 20060249541) discloses a fluid dispensing device includes a bottle for receiving fluid, a discharge tube, and a pressurizing device coupled between the bottle and the discharge tube, for pressurizing the fluid and for forcing the fluid to flow through the discharge tube without gravity. The pressurizing device includes a container coupled between the bottle and the discharge tube, a piston slidably received in the container, and a moving device for moving the piston in a reciprocating action within the container. For example, a motor is coupled to the piston with a crank, to move the piston in the reciprocating action within the container.

Patent application no. U.S. Ser. No. 10/810,686 (US Pub. No. 20050215850) discloses A syringe pump including a syringe including a plunger that slides in a body which has a discharge port, a driving mechanism coupled to the syringe, including a cylinder in which a piston mounted on a shaft slides, and a biasing device operative to apply an urging force on the piston to drive the piston distally in the cylinder, and a safety catch that initially prevents the biasing device from moving the piston, the safety catch being removable to permit the biasing device to move the piston.

FIG. 1 shows an example of an injection system 100 for injecting a medical liquid from a medical liquid container 104 into a common line 102. Those skilled in the art will appreciate the medical liquid container 104 can be any type of container specifically adapted for containing medical liquids, for example, such as, but not limited to: vials, bottles, plastic containers, and any type of container manufactured to contain a medical liquid. The injection system 100 comprises a first connector 106 configured to be connected to a medical liquid container 104, an injector 108 to which is attached an injection device 110 with a medical tubing interface 112, a medical liquid supply line 114 configured to connect the first connector 106 to the medical tubing interface 112 for supplying the medical liquid to the injection device 110, and a common line 102 configured to be connected to the medical tubing interface 112 and to a patient line 116 for injecting the medical liquid into the patient line 116. More precisely, a first tubing valve 130 connects the liquid supply line 114 to common line 102 and allows only passage to the common line 102 under a vacuum pressure. The common line 102 comprises a second tubing valve 140 downstream of the first tubing valve 130 and only authorizes passage towards the patient line 116. The injection device 110 usually comprises a body defining an inner space, and a piston driven by a plunger rod and configured for travelling within the inner space to pump fluid into the inner space or push the fluid out of the inner space.

In the depicted example, two different types of medical liquids are to be injected into the patient, and consequently, the injection system 100 is configured to be connected to two medical liquid containers 104, with two different first connectors 106 and two different medical liquid supply lines 114. However, the injection system 100 may be configured to inject only one medical liquid. For simplicity's sake, the following description will be made with reference to a configuration where only one medical liquid is to be injected, since bi-injection merely involves replicating the described features. Here “bi-injection” is understood to mean two-injections.

For economic and ecological reasons (less use of plastic), the multi-patient practice is steadily gaining market share. In the so-called multi-patient practice, the tubing for the injection system 100 is provided with two very distinct parts: the day set 120 and the patient set 122. The patient set 122 is changed for each patient. The patient set 122 is typically used to limit risks of cross contamination between consecutive patients and thus protects the day set. Once installed and primed the day set 120 stays connected on the power injector for several patient cases, as long as the same medical fluid is to be injected. When the medical fluid to be injected needs to be changed, then the day set 120 is changed. This day set 120 comprises the medical liquid supply line 114 which is connected to the medical liquid container 104 and to the common line 102. The patient set 122 comprises the patient line 116 which is supplied in medical liquid by the common line 102 and is connected to a catheter or a needle for injecting the medical liquid into the patient.

When the injection system 100 is set up for injecting medical liquid into a patient, it is important to ensure that no gas is present in the tubing before injection. Injecting a gas such as air into a blood vessel of a patient may result in a gas embolism, i.e. a blood vessel blockage caused by one or more bubbles of air or other gas in the circulatory system. When the day set 120 or the patient set 116 is put in place, the tubes are filled with air. It is therefore necessary to evacuate the gas present in the tubes before injection. Due to the length of the tubes, a large quantity of gas is to be evacuated from the injection system 100.

For purging the injection system 100 from all the gas present before the injection, the injection device 110 draws or pulls medical liquid from the medical liquid container 104, thereby filling the medical liquid supply line 114. The injection device 110 is now filled with a mixture of medical liquid and gas. The injection device 110 is then positioned with the medical tubing interface 112 upwards so that the gas is gathered at said medical tubing interface 112. It should be noted the filling of the injection device 110 causes a turbulent flow of medical liquid, generating micro-bubbles in the medical liquid. Due to the high viscosity of the medical liquid (especially for contrast agent), the micro-bubbles may need several minutes for reaching the medical tubing interface 112. It is therefore customary to wait at least 2 or 3 minutes with the medical tubing interface 112 upwards. Then, by actuating the piston, the gas is evacuated from the injection device 110 through the still upwards medical tubing interface 112, into the common line 102. Medical liquid is then injected into the common line 102 for pushing the gas outside the common line 102, thereby purging the injection system 100.

During injection, it may happen that gas is present in the injection device 110. For example, vaporization of the medical liquid may create gas. Also, some bubbles generated during the initial filling of the injection system 100 may be blocked in the tubing or against the walls of the injection device 110 and may not be evacuated during the initial purge. As a result, during injection, the injection device 110 is positioned with the medical tubing interface 112 downwards, so any gas present in the injection device 110 will be trapped in the injection device 110 away from the medical tubing interface 112, and will not be injected into the common line 102.

This approach suffers from several drawbacks. First, the injection device 110 must be moved between two opposite positions: with the medical tubing interface 112 upwards or downwards. This requires the injector 108 be able to rotate. Secondly, this purge takes a significant amount of time, and the injection system 100 must be monitored by an operator during the purge. The operator must also evaluate the quality of the purge and whether the purge has completed or not. As with every human interaction, reliance on an operator may lead to errors. Thirdly, the gas is pushed along the common line 102 by medical liquid which also exits the injection system. This approach thereby involves wasting medical liquid, and requires collecting the wasted medical liquid at the output of the injection system 100, with possible handling error.

Also, gas still present in the injection device 110 after the purge may alter the operating of the injection device, even if the gas is trapped in the injection device 110. Dosage of the medical liquid is usually controlled through the course of the piston of the injection device 110. Gas is compressible, and the volume variation of medical fluid within the injection device will therefore be inaccurate. Also, the volume of gas trapped in the injection device 110 must be small, otherwise it risks being injected into the common line 102.

Accordingly, there is a need for an injection system able to evacuate the gas without requiring any operator's involvement, rapidly and each time gas is present in the injection device.

### SUMMARY OF THE INVENTION

It is proposed an injection device for injecting into a medical tubing a medical liquid from a medical liquid container, said injection device comprising:
a body defining an inner space extending in a longitudinal direction between an upper end of the body and a lower end of the body, the lower end of the body comprising a medical tubing interface through which the medical liquid can penetrate the inner space from the medical liquid container and can exit said inner space to be injected into the medical tubing,
a piston arranged within the inner space and configured for travelling within the inner space along the longitudinal direction, said piston delimiting an upper space and a lower space of the inner space, said lower space configured to receive the medical liquid,
an evacuation path, said evacuation path traversing the piston in the longitudinal direction from the lower space to the upper space of the inner space,
a float configured for floating on the medical liquid and arranged in a cavity within the evacuation path between a lower portion of the evacuation path and an intermediate portion of the evacuation path, said lower portion of the evacuation path being connected to the lower space, said cavity comprising at least a passage which belongs to the evacuation path, wherein said float is configured for travelling within said cavity along the longitudinal direction between a blocking configuration in which an obturating part of the float obturates the passage, thereby closing the evacuation path, and an open configuration in which the obturating part 24a of the float is spaced apart from the passage, thereby letting open the evacuation path,
wherein the float has a metacenter which is spaced apart from the top of the obturating part by a distance less than one third of the height of the float.

Other preferred, although non-limitative, aspects of the invention are as follows, isolated or in a technically feasible combination:
the float is configured to have the metacenter above the floating line between the medical liquid and air when the float floats on the medical liquid;
the metacenter is spaced apart from the top of the obturating part by less than 0.50 cm;
a center of gravity of the float is above the center of buoyancy;
the float is configured to float on the medical liquid with a floating line, and a center of gravity of the float is less than 1.0 cm from a floating line on the float;
the float is configured so that the floating line when the float floats on the medical liquid is at least at a diameter of the float corresponding to 80% of a maximum diameter of the float;
the float has a maximum diameter which is more than five times a maximum diameter of a section of the obturating part which obturates the passage;
the float has a center of buoyancy which is spaced apart from the top of the obturating part by at least 1.0 cm;
a volume of air at least 1.0 mL is trapped in the cavity at the closure of the passage by the obturating part;
the float comprises an upper portion and a bottom portion, wherein the upper portion supports the obturating part of the float and has a diameter which is smaller diameter than the bottom portion or a diameter which decreases from the bottom portion;
in the blocking configuration in which the obturating part of the float obturates the passage, a surface of the bottom portion is between 1.8 mm and 2.8 mm from a surface of the seat;
the upper portion has a convex outer surface toward the obturating part;
the float comprises a keel beneath the bottom portion;

The invention also relates to an injection system comprising:
an injection device according to the invention,
a first connector configured to be connected to a medical liquid container,
a medical liquid supply line configured to connect the first connector to the medical tubing interface for supplying the medical liquid to the injection system,
a common line configured to be connected to the medical tubing interface and to a patient line for injecting the medical liquid into the patient line.

### BRIEF DESCRIPTION OF THE DRAWINGS

Other aspects, objects and advantages of the present invention will become better apparent upon reading the following detailed description of preferred embodiments thereof, given as non-limiting examples, and made with reference to the appended drawings wherein:

FIG. 1 is a general view of an injection system with two medical liquid containers connected to it;

FIG. 2 is a cross-sectional view of a piston of an injection device, during a filling step before the purge;

FIG. 3 is a cross-sectional view of a piston of an injection device, during the beginning of the purge;

FIG. 4 is a cross-sectional view of a piston of an injection device at the end of the purge;

FIG. 5 is a cross-sectional view of a piston of an injection device during the injection step after the purge;

FIG. 6 is a cross-sectional view of a piston showing a float in a blocking position, in accordance with a possible embodiment;

FIG. 7 is a cross-sectional view of a float in accordance with a possible embodiment.

### DETAILED DESCRIPTION OF THE INVENTION

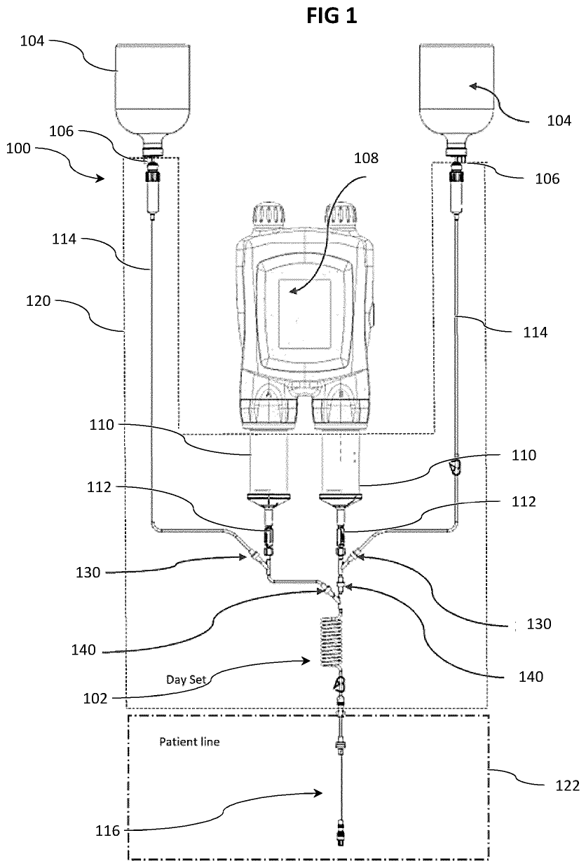

The injection device of the invention can be used in an injection system 100 as previously described in relation with FIG. 1. The injection system 100 will not be described any further, except for the injection device 110.

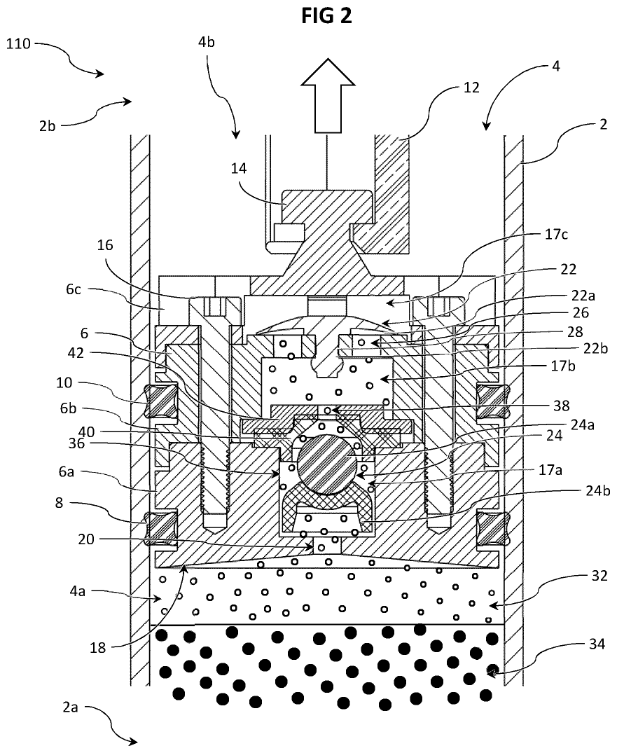

FIGS. 2-5 show examples of an injection device sharing the same features as the claimed invention, except for the design of the float. FIGS. 2-5 and the corresponding passages of the description are provided to allow a better disclosure of the invention, and every feature disclosed in FIGS. 2-5 and the corresponding passages of the description applies to the claimed invention, except for the design of the float.

With reference to FIGS. 2-5, the injection device 110 includes a body 2 defining an inner space 4 extending in a longitudinal direction between an upper end 2b of the body 2 and a lower end 2a of the body 2, the lower end 2a of the body having a medical tubing interface 112 through which the medical liquid can penetrate the inner space 4 from the medical liquid container 104 and can exit said inner space 4 to be injected into the common line 102. Contrary to previous injection devices, the injection device 110 according to the invention is configured for staying with the medical tubing interface 112 downwards. Spatial terms such as “downwards”, “upwards”, “lower”, “upper”, “higher”, “highest” must be understood as defining the commonly accepted relative position with respect to the vertical, i.e. the local Earth gravity direction. This is because gas and medical liquid are subjected to the gravity, and the present invention makes use of the gravity for a proper gas evacuation. As a result, in use, the lower end 2a of the body 2 is located below the upper end 2b of the body 2. The body 2 is typically a hollow cylindric body, for example made of glass or plastic material, also called a barrel.

The injection device 110 also includes a piston 6 arranged within the inner space 4 and configured for travelling within the inner space 4 along the longitudinal direction, i.e. between the upper end 2b of the body 2 and the lower end 2a of the body 2. The piston 6 delimits an upper space 4b and a lower space 4a of the inner space. The lower space 4a is configured to receive the medical liquid, whereas the upper space 4b is not intended to receive any liquid. The piston 6 provides hermetic sealing between the upper space 4b and a lower space 4a. To this end, the piston 6 is provided with at least one peripheral seal 8 or 10, for example made of rubber, and preferably two peripheral seals 8, 10 at different height along the longitudinal direction. Each peripheral seal 8, 10 is pressed against the wall of the body 2, ensuring tight sealing. As in the depicted example, a peripheral seal 8, 10 can be a quad-ring, but can also be for example O-ring.

Due to the tight sealing provided by the piston 6, pressure can significantly differ between upper space 4b and a lower space 4a of the inner space 4. The pressure within the upper space 4b is kept at a reference pressure which is roughly constant and substantially independent from the course of the piston 6. This reference pressure is typically the atmospheric pressure, e.g. the pressure of the system's environment. Preferably, the upper end 2b of the body 2 is at least partially open so the pressure inside the upper space 4b corresponds to the atmospheric pressure, regardless of the course of the piston 6. On the opposite, the pressure inside the lower space 4a of the inner space 4 depends on the content of said lower space 4a and on the course of the piston 6. In the description below, an overpressure is a pressure above the reference pressure, and a vacuum pressure is a pressure below the reference pressure.

The piston 6 is attached to a piston rod 12, for example by a protrusion 14 on top of the piston 6 which is engaged into said piston rod 12. The piston rod 14 is driven by the injector 108 and causes the piston 6 to travel within the inner space 4 along the longitudinal direction. The piston 6 can be formed by several parts assembled together. In the depicted example, the piston 6 has a lower part 6a, an intermediate part 6b and an upper part 6c. Connectors such as screws 16 may be used for assembling the parts of the piston.

The piston 6 has an evacuation path arranged within the piston 6. The path traverses the piston 6 in the longitudinal direction from the lower space 4a to the upper space 4b of the inner space 4. The evacuation path is meant to evacuate into the upper space 4b, the gas present in the lower space 4a. Typically, the evacuation path is not straight and can be closed or open at different points by components of the piston 6, as described below. More specifically, the evacuation path includes a lower portion 17a, an intermediate portion 17b, and an upper portion 17c. The lower portion 17a of the evacuation path is connected to the lower space 4a, whereas the upper portion 17c of the evacuation path is connected to the upper space 4c. The intermediate portion 17b is between the lower portion 17a and the upper portion.

The piston 6 includes a lower interface 18 delimiting the lower space 4a of the inner space 4, said lower interface 18 having an entrance 20 of the evacuation path. Preferably, the entrance 20 opens in a highest portion of the lower interface 18 in order to properly evacuate all the gas present in the lower space, without any gas being trapped in the lower space 4a of the inner space, against the lower interface 18. Preferably, the lower interface 18 has a surface with an apex pointing towards the upper end 2b of the body, and the entrance 20 of the evacuation path opens at said apex. For example, the lower interface 18 has a convex surface seen from the lower space 4a of the inner space, and the entrance 20 of the evacuation path opens in a middle of said lower interface 18, as in the depicted example. For instance, the surface of the lower interface 18 may correspond to a surface of a cone, a truncated cone, or a pyramid pointing upwards. Alternatively, the lower interface 18 may have a concave surface seen from the lower space 4a of the inner space and the entrance 20 of the evacuation path opens in a periphery of said lower interface 18. For example, the lower interface 18 may have a groove arranged in a periphery of the lower interface 18, and the entrance 20 of the evacuation path may open in said groove.

The piston 6 includes a purge valve 22 arranged in the evacuation path between the intermediate portion 17b of the evacuation path and the upper portion 17c of the evacuation path. The purge valve 22 is configured for moving between a blocking configuration in which the purge valve 22 closes the evacuation path, and a passing configuration in which the purge valve 22 keeps open said evacuation path. The passing configuration of the purge valve 22 requires an overpressure in the intermediate portion 17b of the evacuation path with respect to the reference pressure in the upper space 4b, caused by the piston 6 travelling towards the lower end 2a of the body 2. Since the upper space 4b is at the reference pressure (e.g. the atmospheric pressure), the overpressure means a pressure above the reference pressure. More precisely, the overpressure required for the passing configuration of the purge valve 22 corresponds to a pressure in the intermediate portion 17b exceeding the reference pressure in the upper space 4b and the upper portion 17c of the evacuation path by at least a first pressure threshold. The purge valve 22 is configured for being in the blocking configuration when the piston 6 travels towards the upper end of the body 2 since there is no overpressure in the intermediate portion 17a with respect to the reference pressure in the upper space 4b, but instead a vacuum pressure, i.e. a pressure below the reference pressure.

The piston 6 also includes a selector 24 arranged within the evacuation path between the lower portion 17a of the evacuation path and the intermediate portion 17b of the evacuation path. The selector 24 is configured for selectively allowing the gas to go through said selector 24 and to travel along the evacuation path from the lower portion 17a to the intermediate portion 17b of the evacuation path. The selector 24 is also configured for selectively preventing the medical liquid from going through said selector 24 and from travelling along the evacuation path from the lower portion 17a to the intermediate portion 17b of the evacuation path, and consequently from travelling along the evacuation path from the lower space 4a to the upper space 4b of the inner space. As a result, the lower portion 17a of the evacuation path is a mixed portion configured for receiving both medical liquid and gas, and the intermediate portion 17b and upper portion 17c are gaseous portions configured for receiving only gas.

The purge valve 22 is arranged above the selector 24 in the longitudinal direction from the lower space 4a to the upper space 4b of the inner space. The purge valve 22 is thus arranged in the gaseous portion of the evacuation path and is not in contact with any liquid.

In the blocking configuration, the purge valve 22 seals a vent 26 between the intermediate portion 17b and the upper portion 17c of the evacuation path. In the passing configuration, the purge valve 22 lets said vent 26 open. In the depicted example, two vents 26 appear between the intermediate portion 17b and the upper portion 17c of the evacuation path. More or fewer vents 26 can be provided, as long as they are sealable by the purge valve 22.

Preferably, as depicted in the illustrated embodiment, the purge valve 22 is an umbrella valve, which has a diaphragm shaped sealing disk 22a and a stem 22b. The stem 22b is engaged into a hole 28 arranged in a fixed part of the piston 6 and presents an enlarged lower portion with a section superior than the section of the hole 28, thereby affixing the purge valve 22. The diaphragm shaped sealing disk 22a is disposed above at least one vent 26 which is part of the evacuation path and delimits the intermediate portion 17c from the upper portion 17c of the evacuation path. The umbrella valve can be deformable and/or can slidably move along the hole 28 arranged in the fixed part of the piston 6 to change configuration (for example due to the deformation of the stem). In the passing configuration, the sealing disk 22a is away from the vent 26 because of a higher gas pressure within the intermediate portion 17b, thereby allowing gas passing through said vent 26. In the blocking configuration, the sealing disk 22a is pressed against the vent 26 because of a higher gas pressure within the upper portion 17c of the evacuation path, thereby sealing said vent 26, and closing the evacuation path. For example, the umbrella valve can be made of elastomer of rubber type, or in silicon.

The selector 24 is a float 24 configured for floating on the medical liquid.

A process for operating an injection device with a float 24 as a selector will now be described with reference to FIGS. 2-5.

In FIG. 2, the injection device 110 is shown before the purge, for instance during the filling step wherein the injection device 110 is filled with medical liquid and unwanted gas. During this filling step, the piston rod 12 is driven upwards, for example by the injector 108 acting on said piston rod 12, thereby causing the piston 6 to travel within the inner space 4 along the longitudinal direction towards the upper end 2b of the body 2, i.e. upwards. The lower space 4a expands, resulting in a gas pressure within said lower space 4a to drop below the reference gas pressure within the upper space 4b, e.g. the atmospheric pressure.

Since the selector 24 allows gas to travel from the lower portion 17a to the intermediate portion 17b of the evacuation path, the decreased pressure in the lower space 4a is also found in the lower portion 17a and the intermediate portion 17b of the evacuation path. However, as the purge valve 22 requires an overpressure in the intermediate portion 17b of the evacuation path to be in the passing configuration, the purge valve 22 is kept in the blocking configuration. More precisely, the purge valve 22 is pressed downwards, sealing the vent 26 and thereby closing the evacuation path in the blocking configuration.

The expansion of the lower space 4a combined with the closing of the evacuation path by the purge valve 22 effectively creates a vacuum pressure in the lower space 4a, i.e. a pressure below the reference pressure. The pressure in the lower space 4a decreases until reaching the opening pressure of the first tubing valve 130, which is, for example, comprised between 0.2 and 0.5 bars below the reference pressure. The opening of the first tubing valve 130 creates an aspiration of the medical liquid to compensate this vacuum pressure in the lower space 4a. The medical liquid travels through the filling line 114 connected to the medical liquid container 104 to fill the lower space 4a. Gas present in the tubing is also aspirated into the lower space 4a.

Progressively, as the lower space 4a becomes filled with medical liquid and gas, the pressure within the lower space 4a increases and becomes closer to the atmospheric pressure. When the pressure within the lower space 4a reaches the closing pressure of the first tubing valve 130 (substantially similar to the opening pressure), the first tubing valve 130 closes and the filling is stopped. At the end of the filling step, the lower space 4a is filled with a volume of gas 32 above a volume of medical liquid 34. The gas pressure within the lower space 4a is still below the gas pressure within the upper space 4b because the pressure increase was stopped by the closing of the first tubing valve 130 before the vacuum pressure had been completely compensated. Consequently, the purge valve 22 stays in the blocking configuration.

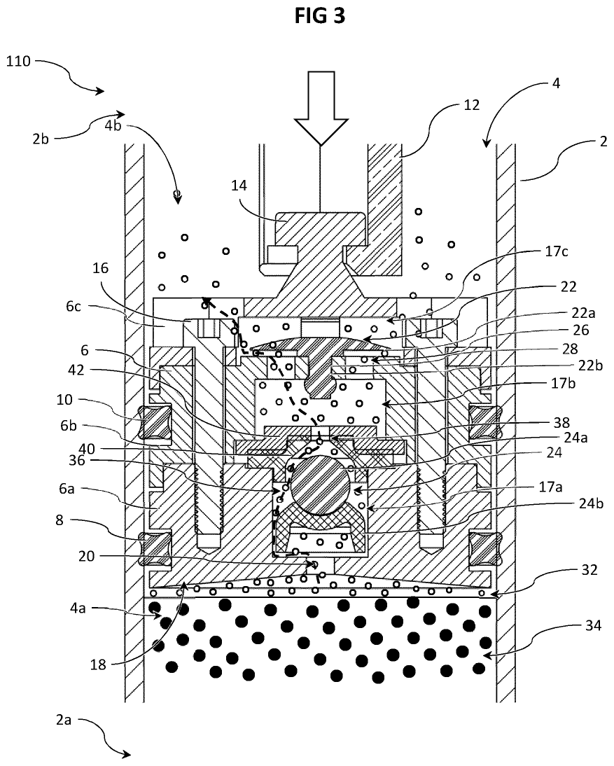

As explained above, after the filling of the injection device 110, the gas in the lower space 4a must be evacuated during a purge. This purge is performed by driving the piston 6 downwards, as shown on FIG. 3. The piston 6 travels within the inner space 4 along the longitudinal direction towards the lower end 2a of the body 2. The lower space 4a shrinks, and gas pressure increases in the lower space 4a until the pressure difference between the gas pressure within the intermediate portion 17b, still communicating with the lower space 4a, and the reference gas pressure within the upper space 4b becomes higher than the opening pressure threshold of the purge valve 22, i.e. until the pressure difference reaches the first pressure threshold.

As indicated above, the purge valve 22 is configured to move to the passing configuration in response to an overpressure in the lower space 4a exceeding the reference pressure in the upper space 4b by at least the opening pressure threshold of the purge valve 22 (for example between 20 and 100 mbars of pressure difference between the overpressure and the reference pressure). The purge valve 22 therefore moves to the passing configuration, thereby opening the evacuation path. In the depicted example, the sealing disk 22a moves away from the vents 26, thereby unsealing said vents 26.

The gas is evacuated from the lower space 4a to the upper space 4b of the inner space 4 through the evacuation path traversing the piston 6. More specifically, gas enters the evacuation path through the entrance 20, then travels along the lower portion 17a of the evacuation path, then along the intermediate portion 17b of the evacuation path, then through the vents 26 and finally along the upper portion 17c to reach the upper space. This is shown by the dotted arrow in FIG. 3.

As the piston 6 travels downwards while the gas is evacuated through the evacuation path, the piston reaches the volume of medical liquid in the lower space 4a. More specifically, the lower interface 18 contacts the medical liquid, and the gas is pushed back towards the entrance 20 of the evacuation path since the entrance 20 opens in a highest portion of said lower interface 18. The gas is therefore evacuated from the lower space 4a before the medical liquid reaches the entrance 20 of the evacuation path. When all the gas has been evacuated, the medical liquid penetrates through the entrance 20 in the lower interface 18 of the piston 6 and fills the lower portion 17a of the evacuation path.

As illustrated, the lower portion 17a of the evacuation path can include a cavity 36 where the selector 24 is arranged and the medical liquid begins filling the cavity 36. The selector is a float 24 configured for floating on the medical liquid and the cavity 36 is configured to allow the float 24 to travel up and down the cavity 36, along the longitudinal direction. The cavity 36 includes at least a passage 38 forming the boundary between the lower portion 17a and the intermediate portion 17b of the evacuation path. The passage 38 is arranged at the top of the cavity 36. The float 24 is configured for travelling within the cavity 36 along the longitudinal direction between a blocking configuration in which the float 24 obturates (i.e., blocks) the passage 38, thereby closing the evacuation path, and an open configuration in which the float 24 is spaced apart from the passage 36, thereby keeping open and unblocked the evacuation path.

More precisely, when the cavity 36 is filled with gas 32, the float 24 stays at the bottom of the cavity 36, keeping open the passage 38 and thereby keeping the evacuation path unblocked. When the medical liquid reaches the cavity 36, the float 24 begins floating on the medical liquid, and therefore moves up, carried by the medical liquid 34 in accordance with the upward buoyant force that is exerted on the float by the medical liquid (Archimedes' principle). Under this force, the float 24 travels upward until reaching the top of the cavity 36 and blocks the passage 38.

The passage 38 is defined by a periphery forming a seat 40 for the float 24, facing said float 24. The float obturates the passage 38 by pressing against the seat. The seat 40 is for example made of metal or plastic such as thermoplastic polyurethane, polyoxymethylene, polycarbonate, polyvinyl chloride, etc. Advantageously, the seat 40 is made from a material with an elastic modulus higher than 2500 Mpa (megapascals). Preferably, the seat 40 has a decreasing section in the direction of the intermediate portion 17b of the evacuation path and, for example, the shape of the seat 40 is, at least partially, a hollow truncated cone. A reinforcing element 42, such as a washer, can be provided above the seat 40 to strengthen the seat, especially when said seat is made in a highly deformable material.

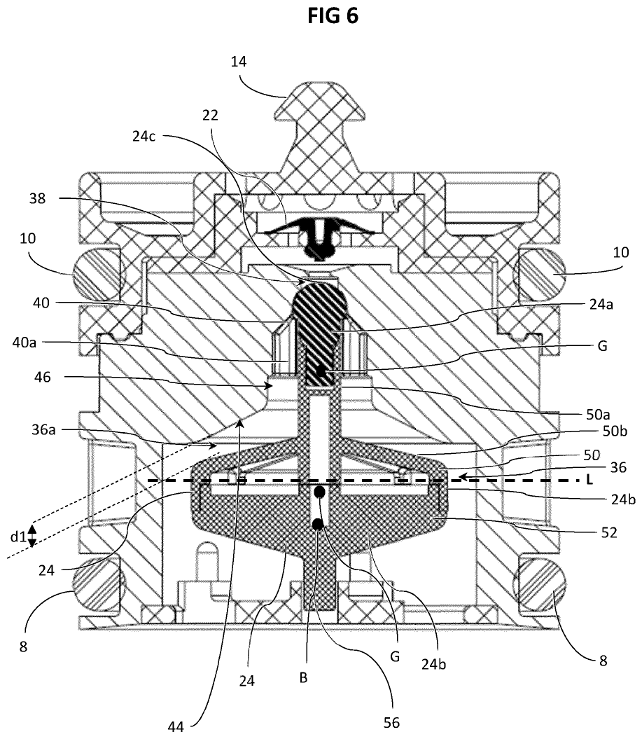

As in the depicted example of FIG. 6, the float 24 has at least an obturating part 24a configured to obturate (e.g., obstruct or block) the passage 38. Typically, the passage 38 has a round section and the obturating part 24a of the float 24 has a round section too. For example, the obturating part 24a of the float 10 may have at least partially an ellipsoid shape, or a spherical shape or a conical shape like a pine. For example, the float 24 can be a mere ball. Preferably, the obturating part 24 of the float is coated with a deformable material for a better sealing of the passage 38 when the float 24 is pressed against the seat 40.

Preferably, the float 24a has a floating part 24b with an enlarged section with respect to a widest section of the obturating part 24a, said floating part 24b supporting the obturating part 24a. The float can be in two-parts as in FIGS. 1-5 or can be in a single piece including the obturating part 24a and the floating part 24b. The obturating part 24a of the float 24 is for example a ball. The floating part 24b of the float 24 has a diameter superior to the widest diameter of the obturating part 24a of the float, i.e. the ball portion. The higher diameter of the floating part 24b of the float 24 (perpendicularly to the longitudinal direction) makes the float 24 more responsive to a low force exerted by the medical liquid 34 on the float 24. This enhanced response allows pushing the float 24 upwards as soon as medical liquid reaches the float 24 and therefore ensures that no medical liquid can reach the passage 38 before said passage is obturated by the float 24.

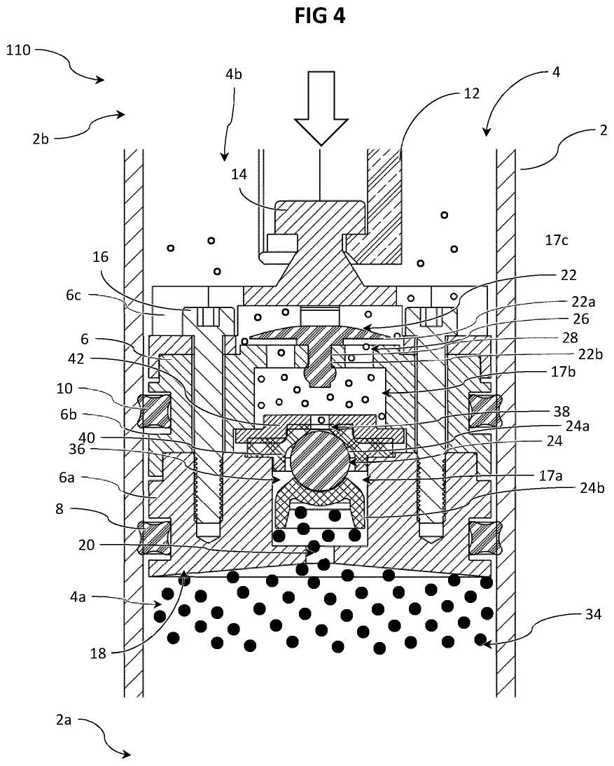

When the medical liquid 34 contacts the float 24, e.g. the floating part 24b of the float 24, the float 24 begins floating and therefore is moved upwards until it reaches the seat 40, as illustrated in FIG. 4. The shape of the float 24 and the shape of the seat 40 are adapted so that a continuous sealing is created when the float 24 presses the seat 40. The passage 38 is now obturated, and neither gas nor medical liquid can pass through the obturated passage 38. A small volume of gas may be trapped within the cavity 36, i.e. in the lower portion of the evacuation path, between the obturated passage 38 and the surface of the medical liquid 34. This small volume enables the seat to be kept dry and to avoid any material deposition on the seat 40. The small volume, for example, may be a volume less than 10 ml at atmospheric pressure. This small volume of air may be a sufficient quantity to dry the float 24. The density of the float 24 and the complementary shapes of the obturating part 24a and of the seat 40 are chosen to minimize the volume of trapped air, while keeping the surface of medical liquid away from the seat 40. For example, the seat's section may decrease in the direction of the intermediate portion 17b of the evacuation path.

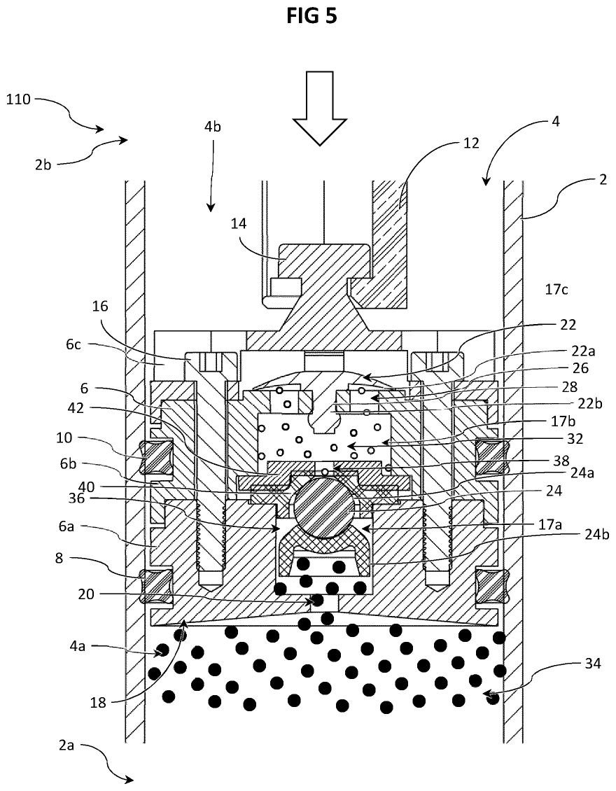

Once the passage 38 is obturated, no gas escapes the overpressure in the lower portion 17a of the evacuation path to reach the intermediate portion 17b. As a result, the pressure within the intermediate portion 17b above the obturated passage 38 drops until the difference between the pressure in the intermediate portion 17b and the reference pressure reaches the closing pressure threshold of the purge valve 22, which is slightly above the reference pressure since the upper space 4b is at said reference pressure. For example, the closing pressure threshold of the purge valve 22 can correspond to a positive pressure difference of 20 to 100 mbars between the pressure within the intermediate portion 17b and the reference pressure. Preferably, the closing pressure threshold and the opening closing threshold are substantially the same, but they can also differ. As a result, the purge valve 22 is now closed (FIG. 5) in the blocking configuration. In this example, the sealing disk 22a covers the vent 26. The purge step is over. A small volume of gas 32 at a residual overpressure (i.e. slightly above the reference pressure) is kept in the intermediate portion 17b. The residual overpressure is not high enough to cause the purge valve 22 to open the vents 26. This residual overpressure ensures that air at the atmospheric pressure coming from the upper space 4b cannot penetrate the intermediate portion 17b of the evacuation path, thus avoiding non-sterile air from entering the sterile area where the medical liquid circulates.

The process may include a complementary filling step which occurs after the purge step and before the injection step. The complementary filling step allows filling the lower space 4a with an accurate prescribed medical liquid volume 34, which was not possible in the first filling step due to the gas volume 32 that led to incorrect volume measures (usually based on the piston 36 course).

The process may include an injection step wherein the piston travels within the inner space along the longitudinal direction towards the lower end 2a of the body, and wherein the medical liquid exits said lower space of the inner space to be injected into a medical tubing. As the evacuation path is closed, pressure increases within the lower space 4a when the piston 6 is pushed downward. When the pressure in the lower space 4a reaches the opening pressure of the second tubing valve 140, the second tubing valve 140 opens and the medical liquid 34 can exit the lower space 4a and travels the common line 102 to reach the patient line 116. The medical liquid can thus be injected without any gas. During the injection, the float 24 contacts the medical liquid.

It should be noted that the opening pressure threshold of the purge valve 22 (i.e. the first pressure threshold) is inferior to the opening pressure threshold (the second pressure threshold) of the second tubing valve 140 so that the purge valve 22 opens before the second tubing valve 140 opens when the gas is to be evacuated. During this injection step however, the obturation of the passage 38 by the selector (the float 24) means that the pressure increases in the lower space 4a but does not increase in the isolated intermediate portion 17b of the evacuation path. The purge valve 22 is thus kept in the blocking configuration.

As explained above, the float 24 transitions between an open configuration in which the obturating part 24a of the float 24, 50 is spaced apart from the passage 38, thereby letting open the evacuation path and a blocking configuration in which an obturating part 24a of the float 24 obturates the passage 38, thereby closing the evacuation path.

Because the float 24 floats on the medical fluid, the obturating part 24a closes the passage 38 before the medical fluid reaches the passage 38. Air is trapped in the cavity 36. Once the obturating part 24a of the float 24 obturates the passage 38, the pressure increases in the cavity 36, which reduces the volume of trapped air, and the medical fluid gets closer to the obturating part 24a. It is preferable to keep medical fluid away from the obturating part 24a of the float. If the obturating part 24a becomes wet, drops of medical fluid may leak in the evacuation path in the long run. Since the float 24 has a low weight, another risk is to have a wet obturating part 24a stuck to the passage 38, for instance due to surface tension. This is especially important in that different medical fluids may have various densities, and that the float 24 should be able to properly work with medical fluid of low density as well as with medical fluid of high density.

In order to avoid any medical liquid from entering the passage 38, and preferably from touching the obturating part 24a, it is desirable to ensure that sufficient air is trapped in the cavity 36, what can be called an air mattress which protects the obturating part 24a. A volume of at least 1.0 mL should be trapped in the cavity 36 at the closure of the passage 38 by the obturating part 24a, and preferably a volume superior to 2.0 mL and possibly more than 3.0 mL. It can be considered that at the closure of the passage 38 by the obturating part 24a, the trapped air is at the atmospheric pressure (around 1013 hPa or 1 atm). The volume of trapped air should not be however too great. Since air is compressible, too high a volume of trapped air may lead to difficulty in managing the pressure within the lower space 4a. Preferably, the volume of trapped air should be below 10.0 mL, and preferably below 5.0 mL at the closure of the passage 38 by the obturating part 24a.

In order to have enough trapped air to keep the obturating part 24a dry, it may be advantageous to reintroduce air in the cavity 36 after a few injections if there is no gas to purge. It is possible to take advantage of the fact that the purge valve 22 may leak air under small negative pressure (for example between 0 and −200 mbars) into the intermediate portion 17b of the evacuation path, below the purge valve 22, and therefore into the cavity 36 through the passage 38. A step of air introduction to reintroduce air into the cavity 36 may be performed from time to time, for example periodically. In this step, a regenerative negative pressure is generated in the intermediate portion 17b of the evacuation path, below the purge valve 22, for example by moving the piston 6 in an upwards direction. The regenerative negative pressure is above the opening pressure, or cracking pressure, of the first tubing valve 130, or filling valve, and is typically above-200 mbars and below −20 mbars, and for example −150 mbars. By doing so, there is no filling of medical liquid but the purge valve 22 leaks air, which comes through the intermediate portion 17b of the evacuation path, and penetrates the cavity 36 through the passage 38 which is no longer blocked by the float 24. Air is thus introduced in the cavity 36 to ensure that enough air is trapped to keep the obturating part 24 dry.

The regenerative negative pressure may not be constant, but is maintained for a certain duration, such as 2 to 10 seconds, depending on the leaking rate of the purge valve 22, to allow enough air to be introduced. For example, the regenerative negative pressure is maintained between 10% and 80% of the opening pressure, or cracking pressure, of the first tubing valve 130, and typically comprises between −10 and −200 mbars, and preferably between 50 and 180 mbars.

The center of buoyancy B of the float 24 may also be considered to ensure the passage 38 becomes blocked while the medical fluid is far from the obturating part 24a. More particularly, the center of buoyancy B of the float 24 should be spaced apart from the obturating part 24a, and more precisely, the float has a center of buoyancy B which is spaced apart from a top of the obturating part 24a by more than half of the height of the float, and preferably by at least two-third of the height of the float. The center of buoyancy B of the float 24 is the center of gravity of the displaced volume of fluid corresponding to the volume of the float 24. It is the center of the volume of the float 24.

In one aspect of the present disclosure, the center of buoyancy B of the float 24 is spaced apart, in the longitudinal direction, from a top 24c of the obturating part 24a by at least 1.0 cm, and preferably by at least 2.0 cm, and more preferably by at least 3.0 cm. Typically, the center of buoyancy B of the float 24 may be at or below the third of the height of the float 24 in the longitudinal direction, from the bottom to the obturating part 24a at the top.

Because of the distance between the center of buoyancy B and the top of the obturating part 24a, the float is pushed early upwards toward the passage 38 by the medical fluid. The obturating part 24a of the float 24 thus obturates the passage 38 while the medical fluid is at a distance from the obturating part 24a, with a volume of trapped air sufficient to protect the obturating part 24a even after high pressure is applied (for instance with superior to 15.0 bars and preferably superior to 20.0 bars.

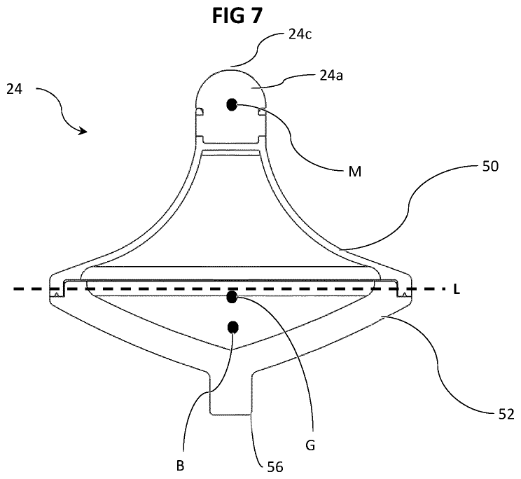

Stability is important, as the obturating part 24a should face the passage 38 to ensure an immediate closure of the passage 38 as soon as the obturating part 24a reaches the level of the passage 38. The float 24 has a metacenter M which is spaced apart from the top of the obturating part 24a by a distance less than one third of the height of the float 24 (along the longitudinal direction), and preferably which is less than one quarter of the height of the float 24. For example, metacenter M is spaced apart from the top of the obturating part 24a by less than 1.0 cm, and preferably less than 0.50 cm. When the float 24 floats on a liquid (for instance with a density of 1, and for instance up to 1.44 or more), the floats 24 may heel, and the center of buoyancy B of the float 24 moves. The metacenter M is the point at which a vertical line through the heeled center of buoyancy B crosses the line through the original, vertical center of buoyancy B is the metacenter M. Preferably, the float 24 is configured to have the metacenter above the floating line between the medical liquid and air when the float 24 floats on the medical liquid. With reference to FIG. 6 and FIG. 7, non-limiting examples of an injection device provided with a float allowing enough air to be trapped are described, in accordance with the claimed invention. FIG. 6 shows an example of a float 24 in a blocking position, obturating the passage 38. FIG. 7 shows another configuration of a float 24, in a cross-sectional view. The float 24 is shaped so that the center of buoyancy B which is spaced apart from the top 24c of the obturating part (in the longitudinal direction). Preferably, the float 24 is at least partially hollow, and preferably is hollower at the top than at the bottom. The top 24c of the obturating part 24a preferably has an at least partially spheroidal shape, and typically is hemispherical.

From top to bottom, the float 24 comprises the obturating part 24a, an upper portion 50 and a bottom portion 52. The upper portion 50 supports the obturating part 24a of the float 24 and has a diameter which is a smaller diameter than the bottom portion 52 and/or a diameter which decreases from the bottom portion 52. The diameter is measured transversally with respect to the longitudinal direction. The bottom portion 52 supports the upper portion 50. The top portion 50 has a diameter which increases from the obturating part 24a to the bottom portion 52. Preferably, the bottom portion 52 is heavier than the top portion 50 and the obturating part 24a.

The upper portion 50 and/or the bottom portion 52 has a maximum diameter larger than the widest diameter of the obturating part 24a of the float. The larger diameter of the bottom portion 52 of the float 24 (perpendicularly to the longitudinal direction) makes the float 24 more responsive to a low force exerted by the medical liquid 34 on the float 24. This enhanced response allows pushing the float 24 upwards as soon as medical liquid reaches the float 24 and therefore ensures that no medical liquid can reach the passage 38 before said passage is obturated by the float 24.

In the example of FIG. 6, the upper portion 50 comprises a downwardly intermediate portion 50b and a stem 50a projecting upwardly from the intermediate portion 50b. Along the longitudinal direction, toward the obturating part 24a, the cross-sectional diameter of the intermediate portion 50b decreases, whereas the cross-sectional diameter of the upper portion 50 is substantially constant. It is also possible to have a continuous diameter decrease along the upper portion 50, as in FIG. 7. In this case, the upper portion may have a convex outer surface toward the obturating part 24a, in order to help evacuating the medical fluid downwards. The diameter of the bottom portion 52 may be at least partially decreasing downwardly.

The float 24 is configured so that the floating line L when the float 24 floats on the medical liquid is at least at a diameter of the float corresponding to at least 80% of a maximum diameter of the float 24, and preferably to at least 90% of a maximum diameter of the float 24, and more preferably is at the maximum diameter. The float 24 has a maximum diameter which is more than two times a maximum diameter of a section of the obturating part 24a which obturates the passage 38, for instance the maximum diameter of a section of the obturating part 24a, and preferably more than five times the maximum diameter of a section of the obturating part 24a. For example, the maximum diameter of the float 24 can be more than 1.5 cm, and preferably more than 2.5 cm. In those examples, the maximum diameter of the float 24 is shared by the upper portion 50 and a bottom portion 52 at their coupling, but this is not a requirement. The maximum cross-sectional diameter of the float 24 may be at the upper portion 50 or at the bottom portion 52 only.

In order to achieve maximal stability, the float 24 is configured such that the floating line or waterline when the float 24 floats on the medical liquid is at a maximum diameter of the float 24 (perpendicularly to the longitudinal direction), or at least is at a diameter corresponding to 80% of the maximum diameter of the float 24. The waterline or floating line here designates the interface between the water taken as medical fluid and the air above.

The higher diameter of the float 24 (perpendicularly to the longitudinal direction) makes the float 24 more responsive to a low force exerted by the medical liquid 34 on the float 24. This enhanced response allows pushing the float 24 upwards as soon as medical liquid reaches the float 24 and therefore ensures that no medical liquid can reach the passage 38 before said passage is obturated by the float 24. Also, the large section of the float 24 increases the force applied by the obturating part 24a in the blocking configuration, and improves the tightness of the sealing of the passage 38.

At least the obturating part 24a may be distinct, and is for example made of a semi-rigid material such as TPE-U, TPE-S, TPE-E, Silicon rubber, Liquid silicon rubber or other material with a hardness shore comprising between 30 ShA and 95 ShA are more advantageously around 70 ShA, whereas the rest of the float 24 is made of a harder material such as like PC, PA, PE, PP, or other raw material with a mechanical resistance higher between 200 et 2500 Mpa and more adv around 800 Mpa. The float 24 may be made of one piece with several materials, such as a dense plastic core at the bottom on which is molded a body of a lower density material such as a foam material. Then the obturating part 24a is molded at the top. It is also possible the float may be assembled from different parts. For example, the upper portion 50 and bottom portion 52 float may be made in one piece, while the obturating part 24a is molded or added at the top. The upper portion 50 and bottom portion 52 may also be distinct, and coupled together, as in the examples of FIG. 7.

The upper peripheral surface 44 of the cavity 36 is the surface of the piston 6 that defines the upper part of the cavity, and leads to the seat 40 where the passage 38 is arranged. The float 24 does not contact the upper peripheral surface 44 of the cavity 36, even in blocking configuration. The upper peripheral surface 44 surrounds an aperture 46 through which a part of the upper section 50, such as the stem 50a, is engaged. The aperture 46 opens to the seat 40. The obturating part 24a goes beyond the aperture 46 to reach the seat 40 and the passage 38. Preferably, the obturating part 24a is always above the aperture 46, even in the open configuration, so that the float 24 is always correctly positioned with the obturating part 24a facing the passage 38.

In order to further guide the float 24 towards the passage 38, the seat 40 may have a guiding portion 40a which surrounds the part of the upper section 50, such as the obturating part 24a and possibly the stem 50a, which is engaged beyond the aperture 46.

In order to improve the efficiency of the trapped air to protect the obturating part 24a, it is desirable that the volume of trapped air results in the medical volume being kept at a distance from the obturating part 24a. Preferably, the upper section 50 of the float 24 and the upper peripheral surface 44 of the cavity 36 have complementary shapes minimizing the volume of trapped air in the clearance 36a between the outer surface of the upper section 50 and the corresponding facing upper surface of the cavity 36 (FIG. 6).

For example, the section of the cavity 36 may decrease in the direction of the passage 38, for instance in the same way as the outer surface of the upper section 50. The clearance 36a must however not be too small, in order to avoid any risk of trapping medical fluid in the clearance 36a, and possibly blocking the movement of the float 24 by capillarity and surface tension phenomena.

Preferably, the minimum or mean distance d1 of the air clearance 36a between the outer surface of the upper section 50 and the corresponding facing upper peripheral surface 44 of the cavity 36 at the closure of the passage 38 is at least 1.8 mm, and preferably at least 2.0 mm. To prevent having too much trapped air, the maximum or mean distance d1 of the air clearance 36a between the outer surface of the upper section 50 and the corresponding facing upper surface 44 of the cavity 36 at the closure of the passage 38 is at most 3.0 mm, and preferably at most 2.7 mm, and more preferably 2.5 mm. Or more generally, most of the distance d1 of the air clearance 36a between the outer surface of the upper section 50 and the corresponding facing upper peripheral surface 44 of the cavity 36 at the closure of the passage 38 is at least 1.8 mm, and preferably at least 2.0 mm. Most of the distance d1 of the air clearance 36a between the outer surface of the upper section 50 and the corresponding facing upper peripheral surface 44 of the cavity 36 at the closure of the passage 38 is at most 3.0 mm, and preferably at most 2.7 mm, and more preferably 2.5 mm. The air clearance 36 preferably has a rotational symmetry around the longitudinal direction. For instance, distances are measured along a normal to the outer surface of the upper section 50 and/or the corresponding facing upper peripheral surface 44 of the cavity 36.

To provide stability, the float 24 should have at least a discrete axial symmetry around the longitudinal axis, and preferably the float 24 has a rotational symmetry around the longitudinal axis. Maintaining the float 24 straight with respect to the longitudinal axis allows the obturating part 24a to always face the passage 38. Accordingly, when the float 24 is pushed upwards by the medical fluid, the obturating part 24a obturates the passage 38 without colliding with the seat 40 surrounding the passage 38. In order to improve the stability of the float 24, the center of gravity G of the float 14 is preferably above the center of buoyancy B.

When the float 24 floats on water (density of 1), and preferably even in a liquid of a density up to 1.44 such as a highly concentrated agent, a center of gravity G of the float 24 is less than 1.0 cm from a floating line (the interface between medical liquid and air), and preferably less than 0.5 cm. Preferably, the center of gravity G of the float 24 is below the waterline. Preferably, the float has a metacentric height (i.e. the distance between the center of gravity G of the float 24 and the metacenter M of the float 24) which is less than 2.0 cm from the top of the obturating part 24a of the float, and preferably less than 1.5 cm.

The upper portion 50 may act as a guide for guiding the obturating part 24a. To further guide the float 24 in the cavity 36, the float 24 may include a keel 56 beneath the bottom portion 52, which extends downwards in the longitudinal direction from a center of the bottom portion 52. The keel 56 has a diameter which is at least five times smaller than the maximum diameter of the bottom portion 52. For example, the keel 56 extends along at least 0.4 cm, and preferably less than 1.0 cm.

While the present invention has been described with respect to certain preferred embodiments, it is obvious that it is in no way limited thereto and it comprises all the technical equivalents of the means described and their combinations. In particular, it will be apparent to those skilled in the art that various changes and modifications may be made without departing from the scope of the invention as defined in the appended claims.

1. An injection device for injecting into a medical tubing a medical liquid from a medical liquid container, said injection device comprising:
a body defining an inner space extending in a longitudinal direction between an upper end of the body and a lower end of the body, the lower end of the body comprising a medical tubing interface through which the medical liquid can penetrate the inner space from the medical liquid container and can exit said inner space to be injected into the medical tubing,
a piston arranged within the inner space and configured for travelling within the inner space along the longitudinal direction, said piston delimiting an upper space and a lower space of the inner space, said lower space configured to receive the medical liquid,
an evacuation path, said evacuation path traversing the piston in the longitudinal direction from the lower space to the upper space of the inner space,
a float configured for floating on the medical liquid and arranged in a cavity within the evacuation path between a lower portion of the evacuation path and an intermediate portion of the evacuation path, said lower portion of the evacuation path being connected to the lower space, said cavity comprising at least a passage which belongs to the evacuation path, wherein said float is configured for travelling within said cavity along the longitudinal direction between a blocking configuration in which an obturating part of the float obturates the passage, thereby closing the evacuation path, and an open configuration in which the obturating part of the float is spaced apart from the passage, thereby letting open the evacuation path,
wherein the float has a metacenter which is spaced apart from a top of the obturating part by a distance less than one third of a height of the float when the float floats on a liquid with a density comprised between 1 and 1.44.

2. The injection device of claim 1, wherein when the float is configured to have the metacenter above a floating line of the float between the medical liquid and air when the float floats on the medical liquid.

3. The injection device of claim 1, wherein the metacenter is spaced apart from the top of the obturating part by less than 0.50 cm when the float floats on the liquid.

4. The injection device of claim 1, wherein a center of gravity of the float is above a center of buoyancy of the float when the float floats on the liquid.

5. The injection device of claim 1, wherein the float is configured to float on the medical liquid with a floating line, and a center of gravity of the float is less than 1.0 cm from a floating line of the float when the float floats on the liquid.

6. The injection device of claim 1, wherein the float is configured so that a floating line of the float when the float floats on the medical liquid is at least at a diameter of the float corresponding to 80% of a maximum diameter of the float.

7. The injection device of claim 1, wherein the float has a maximum diameter which is more than two times a maximum diameter of a section of the obturating part which obturates the passage.

8. The injection device of claim 1, wherein the float has a center of buoyancy which is spaced apart from the top of the obturating part by at least 1.0 cm when the float floats on the liquid.

9. The injection device of claim 1, wherein a volume of air at least 1.0 mL is trapped in the cavity at closure of the passage by the obturating part.

10. The injection device of claim 1, wherein the float comprises an upper portion and a bottom portion, wherein the upper portion supports the obturating part of the float and has a diameter which is smaller diameter than the bottom portion or a diameter which decreases from the bottom portion.

11. The injection device of claim 10, wherein in the blocking configuration the float obturates the passage by pressing against a seat, a surface of the bottom portion is being between 1.8 mm and 2.8 mm from a surface of the seat.

12. The injection device of claim 11, wherein the upper portion has a convex outer surface toward the obturating part.

13. The injection device of claim 1, wherein the float comprises a keel beneath the bottom portion.

14. An injection system comprising:
the injection device of claim 1,
a first connector configured to be connected to a medical liquid container,
a medical liquid supply line configured to connect the first connector to the medical tubing interface of the injection device for supplying the medical liquid to the injection device,
a common line configured to be connected to the medical tubing interface of the injection device and to a patient line for injecting the medical liquid into the patient line.
